(19) United States
(12) Patent Application Publication
(10) Pub. No.: US 2005/0148061 A1
(43) Pub. Date: Jul. 7, 2005

(54) Hybrid plasmid pzzsa coding the synthesis of angiogenis of angiogenin protein and escherichia coli bl21(des) pzzsa strain as the the superoducer of the recombinant chimeric protein of human angiogenin

(76) Inventors: Yury Akhmetovich Ramazanov, Noroskbirsk (RU); Nikolai Pavlovich Metvetsov, Novosibirsk (RU); Velentina Fedorovna Maistrenko, Novosibirsk (RU)

Correspondence Address: LADAS & PARRY, 26 WEST 61ST STREET, NEW YORK, NY, 10023, US

(21) Appl. No.: 10/502,554

(22) PCT Filed: Jan. 30, 2003
(86) PCT No.: PCT/RU03/00049

(30) Foreign Application Priority Data
Jan. 31, 2002 (RU) 2002102856

(51) Int. Cl. C12N 1/21; C12N 15/74

(52) U.S. Cl. 435/252.33; 435/488

(57) ABSTRACT

The invention relates to biotechnology and enhances the expression efficiency of the hybrid gene and stability of the angiogenin protein, as well as provides a possibility of affinity purification of chimeric protein on IgG sorbents.

The hybrid plasmid pZZSA with a molecular weight of 3.814 megadalton (MDa) (6192 bp) which drives the synthesis of the chimeric protein of angiogenin is disclosed, comprising an XhoI/EcoRI fragment of the pGM280 plasmid DNA comprising a tandem of E. coli tryptophan operon promoters, a lambda phage transcription terminator, a β-lactamase gene and an ori replication initiation site; an EcoRI/EcoNI fragment of the PfM plasmid comprising a synthetic translation enhancer of the bacteriophage T7 gene and gene Ap coding for the amino acid sequence corresponding to 3988-4845 bp; synthetic chimeric angiogenin gene (Ang) fused with Spa; unique recognition sites for restriction nucleases.

The strain Escherichia coli BL21 (DE3) pZZSA (the Collection of Microorganisms of the Interregional Center for Human Microcenosis Correction, Novosibirsk, Registration No. MCKM B-127) that is the superproducer of recombinant chimeric protein of human angiogenin is described.

### FIELD OF THE ART

The present invention relates to biotechnology and more particularly to genetic engineering and comprises an in vitro constructed hybrid plasmid pZZSA containing a synthetic gene of angiogenin fused with the gene of the IgG-binding domain of Staphylococcus protein A and Escherichia coli BL21(DE3) pZZSA strain which is the superproducer of chimeric protein of human angiogenin.

Angiogenin is a cationic polypeptide consisting of 123 amino acids with a molecular weight of 14 kDa and is a protein factor of angiogenesis. Angiogenin stimulates the functions of endothelial cells that are important for the development of blood vessels. This accounts for the interest to angiogenin as a promising means for treating wounds, burns, ulcers and cadiovascular pathology.

### PRIOR ART

A process for isolating angiogenin from an HT-9 cells culture medium is known (US 4229331, IPC C12P 1/00, publ. 21 Oct. 1980).

However, the yield of angiogenin is 0.5 μg/l, therefore this source is not effective for producing angiogenin.

A method for isolating angiogenin from blood plasma or serum is known in the art (Bond M. D., Vallee B. L., Isolation of bovine angiogenin using a placental ribonuclease inhibitor binding. Biochemistry, vol. 27, No . 17, pp. 6282-6287, 1988). The yield of angiogenin is 30-80 mg per liter of blood plasma or serum.

A method for angiogenin isolation from cow milk or goat milk is known (RU 2109748, IPC C07K 14/515, publ. 27 Jul. 1998). The yield of angiogenin ranges from 0.27 to 3.27 mg/l.

However, the above-indicated methods utilize costly stock materials, the quantity of which is limited; the purification technology is lengthy, multistep, and requires expensive equipment (HPLC).

A structural gene of human angiogenin synthesized by a chemico-enzymatic method is known. Cloning of this gene in several expressing vector systems, particularly in the genetic construct pRITA16, has been carried out and a number of bacterial strains-producers of hybrids of angiogenin with beta-galactosidase and with Staphylococcus protein A domains have been constructed [Mertvetsov N. P.//Izvestiya Akademii Nauk. Ser. Khim., 1996, vol. 21, No. 12, pp. 2837-2846] and also an E. coli VL1222p R AngT strain-producer has been constructed (deposited with GNII Genetika, Registration No. VKPM V-6895) that produces free angiogenin [Nikonova A. A., Seregin S. V., Chikaev N. A., Mishin V. P., Babkina I. N., Mertvetsov N. P.//Bioorgan. Khimiya, 1996, vol. 22, pp. 891-893].

However, said genetic constructs and bacterial strains are disadvantageous in a low level of the synthetic gene expression amounting to 2 mg of protein per liter of culture medium, this being equivalent to as little as 0.02% of the total cell protein [Studier et al.//Meth. Enzymol., 1990, vol. 185, pp. 60-89].

### DISCLOSURE OF THE INVENTION

The technical result of the proposed inventions is the provision of such vector system and biosynthesis of proteins in the form of hybrids (with a new promoter and a different induction mechanism) and such recombinant E. coli strain-producer which would allow enhancing efficiency of the hybrid gene expression and stability of the angiogenin protein, as well as provide a possibility of affinity purification of chimeric protein on IgG sorbents. Chimeric proteins of such kind per se, with their specific biological activity being preserved, can be used as an active ingredient of medicinal preparations for external use.

Said result is attained by cloning the synthetic gene of human angiogenin fused with the gene of the Staphylococcus protein A IgG-binding domain into Hind III and Xba I restriction sites of a new vector system pET-21 a-d(+) Vectors [8]. As a result, plasmid pZZSA is obtained, in which the hybrid gene is under control of the T7 phage early promoter. The obtained plasmid pZZSA is introduced in cells of E. coli BL21 (DE3) (from the collection of VNII Genetika). The obtained recombinant strain E. coli BL21 (DE3) pZZSA is deposited with the Collection of Microorganisms of the Interregional Center for Human Microcenosis Correction under Registartion No. MCKM B-127 (the Deposition Certificate is enclosed). Protein synthesis in the resulting strain is effected by inducing 1 mM isopropyl β-D-thiogalactoside (IPTG). The strain does not have omp T proteinase of the external cell membrane, which rules out protein degradation in the process of purifying the target product.

The hybrid plasmid pZZSA that drives the synthesis of the chimeric protein of angiogenin is characterized by the following features:
it has as molecular weight of 3.814 megadalton (MDa) (6192 bp);
it codes for the chimeric angiogenin protein;
it consists of:
an XhoI/EcoRI fragment of the plasmid pGM280 (3720 bp) DNA comprising a tandem of E. coli tryptophan operon promoters, a lambda phage transcription terminator, a bla β-lactamase gene and an ori replication initiation site;
an EcoRI/EcoNI fragment of the plasmid PfM (2500 p.o.) comprising a synthetic translation enhancer of the bacteriophage T7 gene, and gene Ap coding for the amino acid sequence corresponding to 3988-4845 bp;
a tandem of E. coli tryptophan operon promoters;
synthetic chimeric angiogenin gene (Ang) fused with Spa;
a selective marker, beta-lactamase gene bla determining ampicillin resistance of E. coli cells transformed with the plasmid pZZSA;
unique recognition sites for restriction nucleases located at the following distances to the right of the EcoRI site (192 bp) with the following coordinates: EcoRI, 192 bp; XbaI, 276 bp; Bgl II, 342 bp; Sph I, 539 bp; EcoNI, 599 bp; Mlu I, 1064 bp.

An essential difference of the proposed plasmid construct is that the hybrid (synthetic) gene of angiogenin is under control of the phage T7 early promoter, whereby providing specific induction of the product (chimeric angiogenin) by isopropyl thiogalactoside and giving a high yield of the target protein, compared with the strains constructed earlier, E. coli JM103 pRITA16 and E. coli VL1222 pRAngT [Mertvetsov N. P.//Izvestiya Akademii Nauk, Ser. Khim., 1996, vol. 21, No. 12, pp. 2837-2846].

For obtaining a strain which is the superproducer of recombinant chimeric protein of human angiogenin, Escherichia coli BL21 (DE3) competent cells are transformed with the recombinant hybrid plasmid pZZSA. The resulting strain Escherichia coli BL21(DE3) pZZSA is characterized by the following features:

Morphological features. Small cells, 1 μm×3-5 μm in size, rod-shaped, gram-negative, non-sporiferous, motile.

Cultural features. When grown on solid LA medium colonies are round-shaped, smooth, semi-transparent, glossy, gray, edges are even; diameter of the colonies is 1 to 3 mm; paste-like consistency. Growth on liquid media (LB, minimum medium with glucose) is characterized by even turbidity with formation of a slight precipitate.

Physico-biochemical features. Cells grow at a temperature of from +4 to +42° C. with an optimum pH of from 6.8 to 7.2. Both mineral salts in ammonium form and organic compounds in the form of peptone, tryptone, yeast extract, amino acids are used as the source of nitrogen. As the source of carbon use is made of amino acids, glycerin, carbohydrates.

Resistance to antibiotics. Cells display resistance to ampicillin (up to 300 μg/ml) determined by the presence of β-lactamase gene in the plasmid, and resistance to tetracycline (up to 50 μg/ml) associated with the presence of transposon.

The obtained strain Escherichia coli BL2(DE3) pZZSA provides IPTG (isopropyl-β-thiogalactoside)-induced constitutive synthesis of chimeric protein of human angiogenin amounting to at least 5% of the total cell protein, this being tenfold that in the prototype.

### BRIEF DESCRIPTION OF THE DRAWINGS

FIG. 1 is a physical map of the constructed recombinant plasmid pZZSA containing the hybrid gene of angiogenin.

Legend to figure:
MCS is a polylinker;
Lac-I is a gene repressing the expression prior to IPTG induction;
Bla is a gene coding for β-lactamase (a selective marker);
Lac O is an operator;
T7 is a promoter;
Ang is an inserted chimeric gene of angiogenin, fused with Spa.
FIG. 2 is a nucleotide sequence of the angiogenin gene comprised by the plasmid pZZSA.

Legend to figure:
EcoRI, XhoI, NcoI, BamHI, SalGI, PstI are restriction endonucleases.

FIG. 3 shows the dynamics of growth of the biomass of the producer E. coli BL21 (DE3) pZZSA in the process of culturing, wherein: 1 is pH of the medium; OD is the optical density of the culture at 590 nm; 2 is the volume of the nutrient medium in the bioreactor; 3.5 liters; 3—2.7 liters. The moment of addition of an inducer (IPTG) is indicated with dotted lines.

FIG. 4 shows the growth curve of the strain E. coli BL21 (DE3) pZZSA in a “BIOK” vortex fermenter.

FIG. 5 shows data (electrophoretic pattern: separation in 12% PAAG) of restriction analysis of recombinant plasmid DNAs—pAng1, pAng2 and pAng3 obtained in a “BIOK” vortex bioreactor: 1, 9—phage λ DNA digested with PstI; 2—pAng1 plasmid DNA; 3—pAng1 plasmid DNA digested with EcoRI; 4—pAng2 plasmid DNA; 5—pAng2 plasmid DNA digested with EcoRI; 6—pAng3 plasmid DNA; 7—pAng3 plasmid DNA digested with EcoRI; 8—pBluescript plasmid DNA digested with MspI.

FIG. 6 shows data (electrophoresis in 15% denaturing PAAG) of chimeric protein analysis in the process of washing off inclusion bodies: 1, 2—ultrasonicated lysate after removal of soluble cell proteins; 3, 4—solubilized proteins in 6M guanidine chloride; 5—molecular weight marker: 14.2 kDa; 20.0; 24.0; 36.0; 45.0; 66.0 kDa.

### VARIANTS OF CARRYING OUT THE INVENTION

### EXAMPLE 1

### A Method for Constructing Plasmid pZZSA

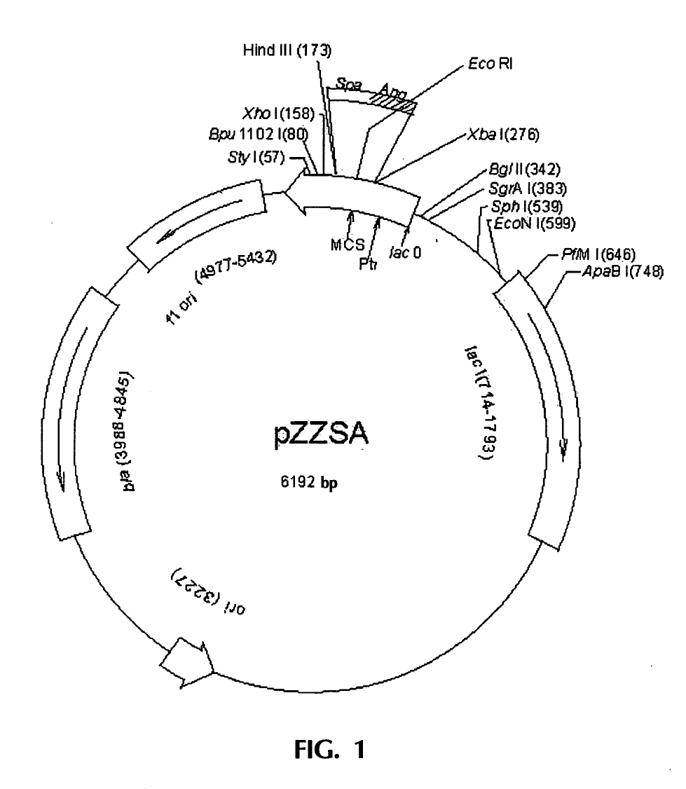

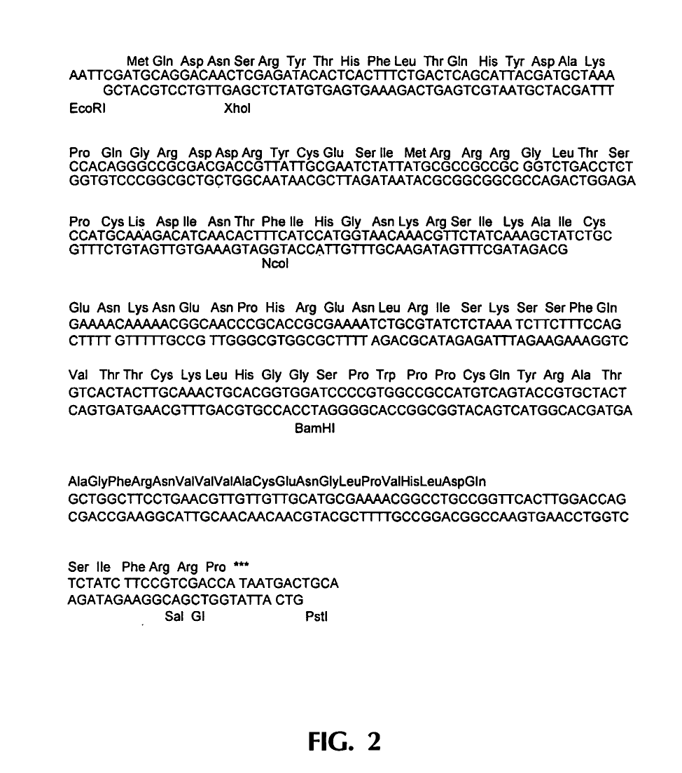

Synthetic gene of angiogenin (having the nucleotide sequence shown in FIG. 2) fused with the gene of Staphylococcus protein A IgG-binding domain (constructed earlier) was cloned into Hind III and Xba I restriction sites the vector pET21a-d(+). As a result, plasmid pZZSA is obtained, in which the hybrid gene is under control of the phage T7 early promoter. The physical map of the constructed recombinant plasmid pZZSA with the hybrid gene of angiogenin is presented in FIG. 1.

When the constructed plasmid pZZSA is introduced into E. coli BL21(DE3) cells protein synthesis in the resulting strain is effected by inducing with 1 mM IPTG. The strain does not have omp T proteinase of the external cell membrane, which rules out protein degradation in the process of purifying the target product.

### Construction Steps

Synthetic gene of angiogenin fused with the gene of the Staphylococcus protein A IgG-binding domain (constructed by us earlier) was cloned into Hind III and Xba I restriction sites the vector pET21a-d(+). As a result, plasmid pZZSA is obtained, in which the hybrid gene is under control of the phage T7 early promoter. When the constructed plasmid pZZSA is introduced into E. coli BL21(DE3) cells protein synthesis in the resulting strain is carried out after inducing with 1 mM IPTG. The strain does not have omp T proteinase of the external cell membrane, which rules out protein degradation in the process of purifying the target product.

### EXAMPLE 2

### Production of Strain E. coli BL21(DE3) pZZSA—Superproducer of Recombinant Chimeric Protein of Human Angiogenin

E. coli BL21(DE3) competent cells are transformed with the pZZSA plasmid and the E. coli BL2 1 (DE3) pZZSA strain that is the superproducer of recombinant protein of human angiogenin is obtained.

### Transformation Procedure

Transformation was carried out using calcium chloride. 1 ml of an overnight bacterial culture was diluted with 100 ml of L-broth. Cells were grown at 37° C. for 4 hours with intense stirring to the density of 5·10⁷ cell/ml. The culture was cooled on ice (10 min.), cells were centrifuged at 4000 g for 5 minutes at 4° C. The supernatant was removed. The cells were resuspended in half the initial volume of ice-cold sterile solution of 50 mM CaCl₂+10 mM Tris-HCl, pH 8.0 The cell suspension was placed into an ice bath for 15 minutes, centrifuged, the supernatant was removed. The cells were resuspended in 1/15 the initial volume of ice-cold sterile solution of 50 mM CaCl₂+10 mM Tris-HCl, pH 8.0.

The cell suspension was placed into an ice bath for 15 minutes, centrifuged, the supernatant was removed. The cells were resuspended in 1/15 the initial volume of ice-cold sterile solution of 50 mM CaCl₂+10 mM tris-HCl, pH 8.0.

0.2 aliquots were poured into pre-chilled test tubes. 40 ng of DNA in 100 ml of ligase buffer was added, mixed, and the mixture was incubated on ice for 30 min. Samples were transferred into a 42° C. water bath for 2 min. 1 ml of L-broth was added into each test tube and incubated for 60 minutes at 37° C. Cells were plated onto 1.5% agar, incubated at 37° C. for 16 hours. Selection for ampicillin resistance was carried out.

### EXAMPLE 3

### Procedure for Culturing Strain of E. coli BL21(DE3) pZZSA and Induction of Protein of Human Angiogenin

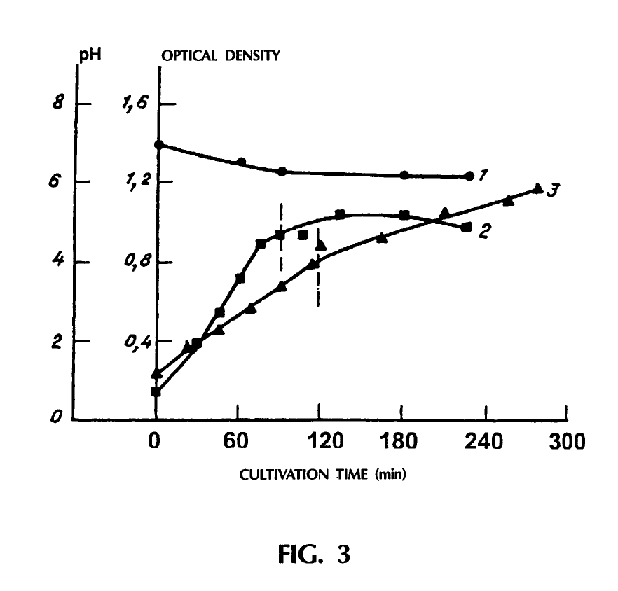

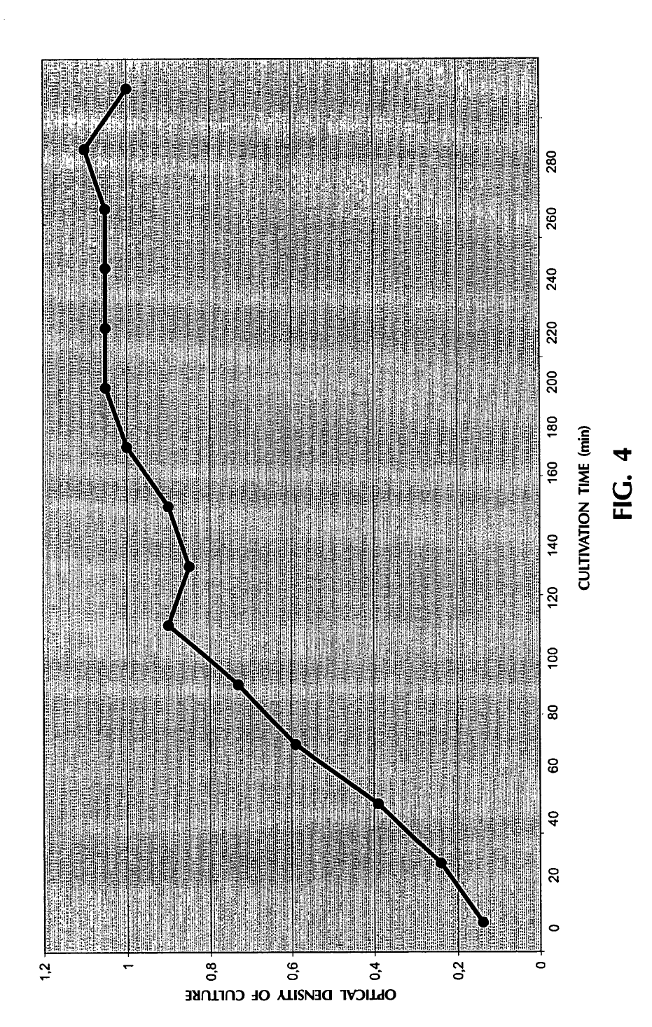

An overnight culture of strain E. coli BL21(DE3) pZZSA is diluted 50-fold in LB medium supplemented with 0.1 mg/ml ampicillin and cultered with aeration in a “BIOK” vortex fermenter (RU 2135579, IPC C12M 1/04, publ. 27 Aug. 1999) in a volume of from 2.5 to 5.0 liters at 37° C. until the logarithmic phase of growth is completed, i.e. when optical density of 1 a. u. at a wavelength of 600 nm is reached. Further, IPTG is added to the suspension to 1 mM concentration, and incubation is continued for additional 3.5 hours to achieve expression of the recombinant gene of angiogenin. The dynamics of growth of the culture of bacterial cells is shown in the diagram (FIG. 4). Upon completion of the incubation, the bacterial cells are collected by centrifugation at 5000 rpm, and then stored at −20° C. till the moment of lysis. The results of cultivation are presented in FIGS. 3 and 4.

### EXAMPLE 4

### Procedure for Isolating Inclusion Bodies and Producing Chimeric Protein of Human Angiogenin

Cells are resuspended in buffer “A” (0.1 M tris-HCl; pH7.2; 0.1 M NaCl; 10 mM beta-mercaptoethanol, 10% sucrose), sonicated using an UZDN2T ultrasonic generator, and centrifuged for 15 minutes at 5000 rpm. The pellt is washed 5 times with buffer “A” supplemented with 1% Triton X-100, 2 M urea, with mandatory homogenization and subsequent centrifugation for 15 min. After removing the supernatant, the pellet is solubilized in 50 ml of 7M guanidine chloride.

Further, for isolating chimeric protein of angiogenin, 2.5 ml of 1M Tris-HCl pH 8.0; 0.5 g of Na tetrathionate, 1 g of Na sulfite, are added to 50 ml of solubilized protein and the resulting mixture is incubated for 20 hours at 22° C. After dialysis against 1 liter of phosphate buffer, pH 8.0, the solution is diluted 10-fold with the same buffer supplemented with 1 mM reduced glutathione and 0.1 mM oxidized glutathione, and incubated for 17 hours at +4° C. After that, DE-52 cellulose chromatography is performed, with a stepwise elution of protein with NaCl solution. The eluent concentration on exit of the protein from the column was 0.5 M NaCl. The yield of the protein is 40 mg per 5 g of the biomass.

### EXAMPLE 5

### Electrophoretic Analysis of Proteins and Analysis of the Specific Activity of Chimeric Protein of Angiogenin

Electrophoretic analysis of proteins is carried out in 15% polyacrylamide gel (PAAG) in Laemmly's buffer system [Laemmly U. K.//Nature, 1970, vol. 227, pp. 680-685]. The degree of purity of the preparation of chimeric protein of angiogenin after purification is evaluated based on the densitometric analysis of the electrophoretic pattern.

The content of chimeric protein in the cell in the process of cultivation is analyzed in a similar manner.

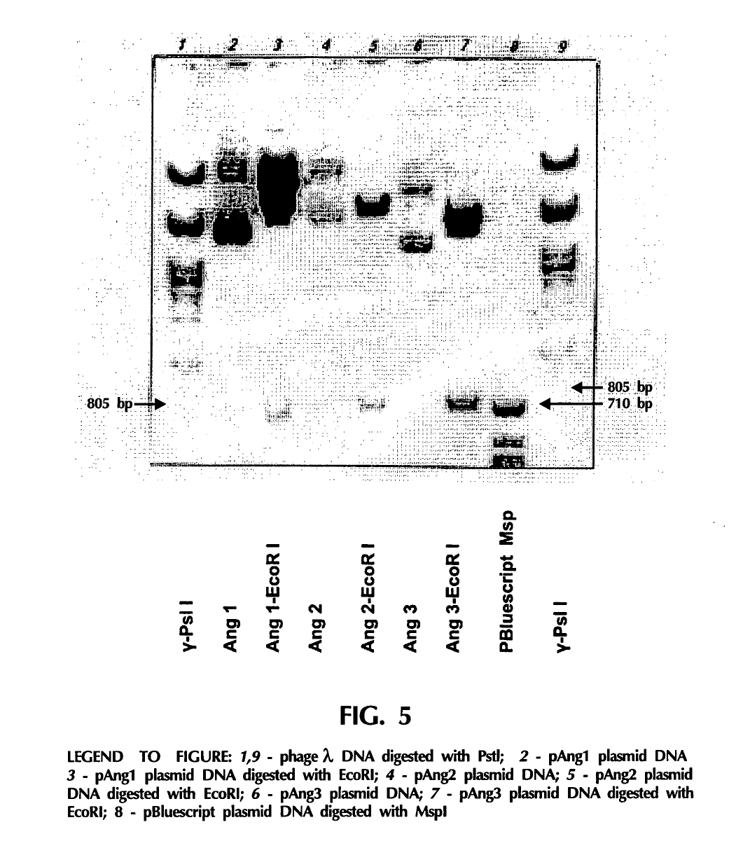

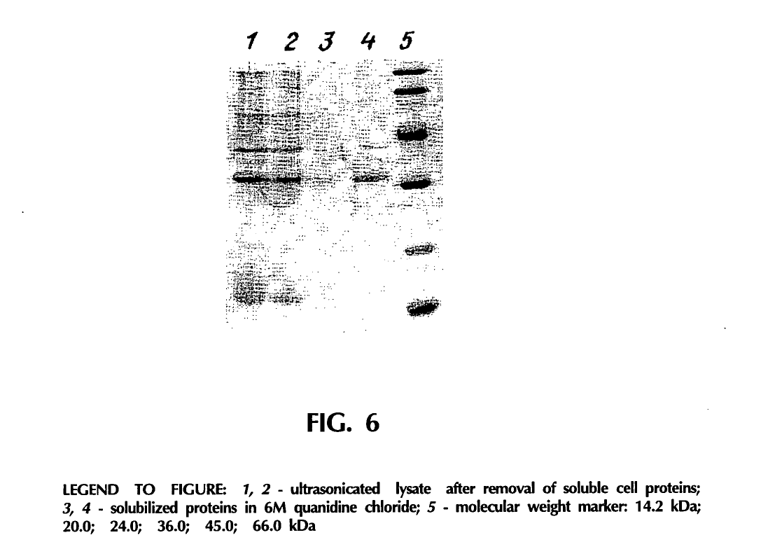

Experiments have shown that the obtained hybrid plasmid pZZSA actually provides expression of the chimeric gene of angiogenin in E. coli cells. According to the data of PAAG electrophoresis the molecular weight of the recombinant protein is 28.8 kDa (FIGS. 5 and 6), 14.1 kDa of which being accounted for by angiogenin [Mertvetsov N. P.//Izvestiya Akademii Nauk, Ser. Khim., 1996, vol. 21, No. 12, pp. 2837-2846].

The dynamics of the IPTG-induced synthesis of angiogenin fused with the Staphylococcus protein A IgG-binding domain is determined by electrophoretic method. According to the data of densitometry of the recombinant protein bands, the yield of the recombinant protein is 20 to 30%. The electrophoretic purity of the protein is about 95%.

Analysis of the specific activity of the recombinant protein is carried out on the chorioallantoic membrane (CAM) of chick embryo [Klasbum M., Knighton D., Folkman J.//Cancer Research, 1976, vol. 36, 1976, pp. 110-114. Fett J. W., Strudom D. J., Lobb R. R., Alderman E. M.//Biochemistry, 1985, vol. 24, pp. 5480-5486] according to the following procedure using 7-9 days old chick embryos with normal development of the fetus.

At least 7 chick embryos per point are taken for the experiment. At least two dilutions of the recombinant angiogenin preparations and two controls are tested in parallel. With observing sterility conditions, the samples to be tested in an amount of 10 μl each are applied to Millipore filters (10 mm diameter disks). The disks are dried briefly under a lamp in a laminar box. Under sterile conditions the filters impregnated with samples of the preparations being tested are applied to CAM. All eggs used in the experiment are marked and placed into a thermostat set at the temperature of 37° C. for further incubation.

The results are read 72-80 hours after the filter with the applied sample of the recombinant angiogenin preparation was implanted on CAM. The eggs with chick embryos are preliminarily analyzed using an ovoscope, and dead ones are rejected. The eggs with live chick embryos are opened up, and visual examination of the vascular network of the chorioallantoic membrane is carried out, including both its part adjacent to the implanted filter disk and the parietal part (adjacent to the shell). Then, a portion of the chorioallantoic membrane is extracted from under the filter disk and placed for 5-7 minutes into formalin solution for fixing. The fixed portions of CAM are placed under a magnifying glass (or under a microscope) and the number of the vessels is counted. The mean value of the measured density of the vessels on 7 embryos for each sample is compared with the degree of vascularization of the chorioallantoic membrane in control samples. The result is scored as positive when the mean value of the density of vessels in the experiment is 2 and more times that in control.

The results of analysis of the angiogenic activity of chimeric protein on CAM of developing chick embryo are presented in Table 1 below.

TABLE 1
Data on angiogenic activity of chimeric protein
on CAM of developing chick embryo

| | | Number of vessels |
|---|---|---|
| | | per unit surface (cm²) |
| | | counted directly |
| Nos. | Preparation | under magnifying glass |
| 1 | 0.9% NaCl (control) | 38.7 ± 12.3 |
| 2 | Protein extract from E. coli free | 36.7 ± 9.7 |
| | of angiogenin plasmid | |
| 3 | Chimeric angiogenin (0.1 mg/ml) | 111.8 ± 85.2 |
| 4 | Chimeric angiogenin (0.2 mg/ml) | 72.6 ± 21.4 |

An analysis of Table 1 shows that the specific activity of the tested samples is positive, since the mean value of the density of vessels in the experiment is 2 and more times that in control [Shapiro R., Strudom D. J., Olson K. A., Vallee B. L.//Biochemistry, 1987, vol. 26, pp. 5141-5146].

### EXAMPLE 6

### Data on Using Chimeric Protein of Angiogenin in Medicine

Chimeric protein of angiogenin obtained from the cell biomass of E. coli BL21 (DE3) pZZSA in accordance with Example 4 is used, for instance, in an ointment form based on polyethylene oxide gel.

The elucidated indications for use in skin diseases and lesions of different etiology:
in bums (frostbites) of different severity, a gel with angiogenin is effective as a first aid as well as in the process of treatment and rehabilitation;
in traumatic injuries of the cutaneous integument in the form of cuts, scratches, cracks, bedsores;
in diseases associated with blood circulation disturbances, trophic ulcers, in cases of varicose veins, obliterating endarteritis of lower extremities;
in skin diseases (sclerodermatitis, psoriasis, non-infectious dermatites, alopecia.

Using an applicator, a thin gel layer is applied on the injured surface in 6-12 hours, and later on, as the wound heals, once a day, till the skin defect is completely remedied.

Long-term experimental studies show that the ointment has a specific healing effect which promotes rapid restoration of the epithelial integument without formation of scars, improves blood circulation in the region of injured tissues owing to restoration of the capillary network, and stimulates local immunity. The gel forms a protective film on the burn or wound surface, whereby the probability of infectious complications, intumescence and painful sensations is significantly lowered.

### INDUSTRIAL APPLICABILITY

The proposed invention may be widely used in medical industry for manufacturing preparations based on angiogenin protein as shown in the above Examples.

1. A hybrid plasmid pZZSA with a molecular weight of 3.814 megadalton (MDa) (6192 bp) that drives the synthesis of the chimeric protein of angiogenin, comprising:
an XhoI/EcoRI fragment of the pGM280 plasmid (3720 bp) DNA comprising a tandem of E. coli tryptophan operon promoters, a lambda phage transcription terminator, a bla β-lactamase gene and an ori replication initiation site;
an EcoRI/EcoNI fragment of the PfM plasmid (2500 bp) comprising a synthetic translation enhancer of the bacteriophage T7 gene, and gene Ap coding for the amino acid sequence corresponding to 3988-4845 bp;
a tandem of E. coli tryptophan operon promoters;
synthetic chimeric angiogenin gene (Ang) fused with Spa;
a selective marker, beta-lactamase gene bla determining the ampicillin resistance of E. coli cells transformed with the plasmid pZZSA;
unique recognition sites for restriction nucleases located at the following distances to the right of the EcoRI site (192 bp) with the following coordinates: EcoRI, 192 bp; XbaI, 276 bp; Bgl II, 342 bp; Sph I, 539 bp; EcoNI, 599 bp; Mlu I, 1064 bp

2. A strain Escherichia coli BL21(DE3) pZZSA (deposited with the Collection of Microorganisms of the Interregional Center for Human Microcenosis Correction, Novosibirsk under Registartion No. MCKM B-127) which is the superproducer of recombinant chimeric protein of human angiogenin.
